(19) United States
(12) Patent Application Publication
(10) Pub. No.: US 2026/0146930 A1
(43) Pub. Date: May 28, 2026

(54) PA Plastic Failure Prediction Method

(71) Applicant: China Special Equipment Inspection & Research Institute, Beijing (CN)

(72) Inventors: Tingting TANG, Beijing (CN); Bo ZHAO, Beijing (CN); Lixian WANG, Beijing (CN)

(73) Assignee: China Special Equipment Inspection & Research Institute, Beijing (CN)

(21) Appl. No.: 19/385,695

(22) Filed: Nov. 11, 2025

(30) Foreign Application Priority Data
Nov. 25, 2024 (CN) 202411690839.2

(51) Int. Cl. G01N 3/32; G01N 3/08; G01N 33/44

(52) U.S. Cl. CPC G01N 3/32; G01N 3/08; G01N 33/442; G01N 2203/0069

(57) ABSTRACT

A polyamide (PA) plastic failure prediction method is provided, including: preparing a PA plastic sample, and configuring a stretching speed and a number of stretches; collecting, by a high-precision acoustic emission monitoring system, in real time during a stretching test, an acoustic emission signal generated by the PA plastic sample under an external load; processing and analyzing collected acoustic emission signal through an acoustic emission analysis software, to identify a characteristic and a pattern of the acoustic emission signal; performing correlation coefficient analysis on the collected acoustic emission signal to determine an evaluation factor, and then determining a failure membership degree of each comment set through a triangular membership degree function; and establishing a PA plastic failure prediction model for principal component analysis based on characteristic parameters of the acoustic emission signal.

### CROSS REFERENCE TO RELATED APPLICATION

This patent application claims the benefit and priority of Chinese Patent Application No. 202411690839.2 entitled “PA PLASTIC FAILURE PREDICTION METHOD” filed on Nov. 25, 2024, the disclosure of which is incorporated by reference herein in its entirety as part of the present application.

### TECHNICAL FIELD

The present disclosure relates to the technical field of polyamide (PA) plastic test methods and systems, and in particular to a PA plastic failure prediction method.

### BACKGROUND

PA plastic has characteristics such as excellent mechanical properties, relatively good electrical properties, strength and toughness, wear resistance, and self-lubrication. With rapid development and industrialization of hydrogen fuel batteries and electric vehicles, IV hydrogen storage cylinders, due to their lightweight and fatigue-resistant characteristics and the like, become a global research hotspot and represent a future application trend. Polyamide (PA) is a preferred material for liners of IV hydrogen storage cylinders, and is one of the most critical materials for hydrogen energy equipment, and serves as a hydrogen barrier. Therefore, researches on PA plastic damage evolution is of significance for structural safety.

Scholars at home and abroad conducted a lot of researches on damage and destruction behaviors on non-metallic materials. ZHAO Wenzheng and other researchers conducted in-depth researches on damage evolution and failure processes of glass fiber-reinforced composite materials under a compressive load through a combination of acoustic emission (AE) and digital image correlation (DIC) technologies, which makes full use of complementary advantages of the two technologies to obtain a more comprehensive understanding of material behaviors. ZHANG Mi et al. conducted researches on propagation characteristics of acoustic emission signals in polymer materials by using an acrylic panel as a research subject. SHEN Shuqian et al. conducted researches on online detection of damage of a laminate during impact by using an acoustic emission test technology, and demonstrated that a distribution law of the wavelet packet energy spectrum could effectively characterize the damage types of the laminate. Juhasz et al. conducted finite element-based progressive analysis on a critical load for delamination propagation of a composite material, with their simulation and calculation being subsequently verified through experimental tests. MENG Chao et al. conducted researches on damage type identification and damage modes during damage of glass fiber-reinforced composite materials by using an acoustic emission (AE) technology. Complexity and nonlinear characteristics of the plastic failure and destruction process make numerous variables exhibit interdependencies, causing difficulty in establishing a precise damage prediction model.

### SUMMARY

A technical problem to be solved in the present disclosure is to provide a PA plastic failure prediction method that could improve accuracy and credibility of an analysis result and effectively improve reliability of PA damage degree prediction.

In order to resolve the above technical problem, technical solutions adopted in the present disclosure are as follows: A PA plastic failure prediction method is provided, including steps of:
preparing a PA plastic sample, and configuring a stretching speed and a number of stretches;
collecting, by a high-precision acoustic emission monitoring system in real time during a stretching test, an acoustic emission signal generated by the PA plastic sample under an external load;
processing and analyzing the collected acoustic emission signal through an acoustic emission analysis software, to identify a characteristic and a pattern of the acoustic emission signal;
performing correlation coefficient analysis on the collected acoustic emission signal to determine an evaluation factor, and then determining a failure membership degree of each comment set through a triangular membership degree function;
establishing a PA plastic failure prediction model for principal component analysis (PCA) based on characteristic parameters of the acoustic emission signal, where the model is configured to evaluate a degree of failure and destruction of a material and to predict a failure occurring time; and
predicting a degree of failure and destruction of the PA plastic sample and a failure occurring time by using the PA plastic failure prediction model.

In some embodiments, the method further includes: performing a further test to collect data of stretching damage and destruction tests at different speeds, performing statistical analysis on characteristic point parameters during damage, comparing the data with an evaluation result of a prediction and evaluation model, and verifying the prediction and evaluation model.

Beneficial effects of the above technical solutions are as follows: According to the method of the present disclosure, internal changes of the material during stress are monitored through the acoustic emission technology, analysis and research are conducted on the acoustic emission signals during PA plastic damage at different stretching speeds in combination with fuzzy mathematics, and a fuzzy prediction and evaluation model for PCA is established to hazily predict the stretching failure condition of the PA plastic and evaluate the degree of damage of the PA plastic, so as to provide a reference for damage-degree evaluation and early damage prediction of a structure of the PA material, which could improve accuracy and credibility of the analysis result and effectively improve reliability of the PA damage prediction.

### BRIEF DESCRIPTION OF THE DRAWINGS

The present disclosure is described in further details below in conjunction with drawings and embodiments.

FIG. 1 shows a flowchart diagram of a method according to an embodiment of the present disclosure, where S1 represents preparing a PA plastic sample, and configuring a stretching speed and a number of stretches; S2 represents collecting, by a high-precision acoustic emission monitoring system, in real time during a stretching test, an acoustic emission signal generated by the PA plastic sample under an external load; S3 represents processing and analyzing collected acoustic emission signal through an acoustic emission analysis software, to identify a characteristic and a pattern of the acoustic emission signal; S4 represents performing correlation coefficient analysis on the collected acoustic emission signal to determine an evaluation factor, and then determining a failure membership degree of each comment set through a triangular membership degree function; S5 represents establishing a PA plastic failure prediction model for principal component analysis based on characteristic parameters of the acoustic emission signal, where the PA plastic failure prediction model is configured to evaluate a degree of failure and destruction of a material and to predict a failure occurring time; and S6 represents establishing a PA plastic failure prediction model for principal component analysis based on characteristic parameters of the acoustic emission signal, where the PA plastic failure prediction model is configured to evaluate a degree of failure and destruction of a material and to predict a failure occurring time; and

FIG. 2 shows a diagram illustrating a correlation between an amplitude and a ring count of acoustic emission (AE) characteristic parameters of a PA sample according to an embodiment of the present disclosure.

### DETAILED DESCRIPTION OF THE EMBODIMENTS

Technical solutions in embodiments of the present disclosure are clearly and completely described below in conjunction with drawings in the embodiments of the present disclosure. Apparently, the embodiments described are merely some rather than all of the embodiments of the present disclosure. All other embodiments obtained by a person of ordinary skill in the art based on the embodiments of the present disclosure without creative efforts fall within the scope of the present disclosure.

Numerous specific details are set forth in the following description to facilitate a full understanding of the present disclosure. However, the present disclosure may be implemented in other ways than those described herein, and a person skilled in the art may make similar extensions without departing from the scope of the present disclosure. Therefore, the present disclosure is not limited by the specific embodiments disclosed below.

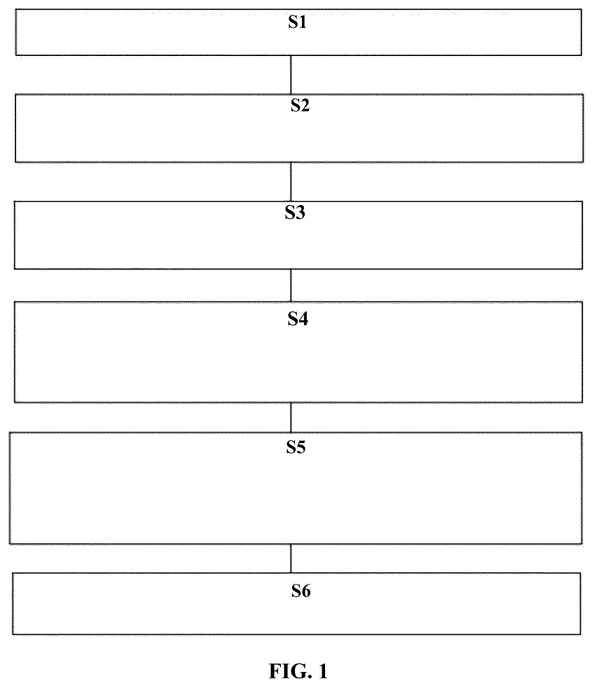

In general, as shown in FIG. 1, an embodiment of the present disclosure discloses a PA plastic failure prediction method, including the following steps:

S1: preparing a PA plastic sample, and configuring a stretching speed and a number of stretches.

S2: collecting, by a high-precision acoustic emission monitoring system, in real time during a stretching test, an acoustic emission signal generated by the PA plastic sample under an external load.

S3: processing and analyzing collected acoustic emission signal through an acoustic emission analysis software, to identify a characteristic and a pattern of the acoustic emission signal.

S4: performing correlation coefficient analysis on the collected acoustic emission signal to determine an evaluation factor, and then determining a failure membership degree of each comment set through a triangular membership degree function.

S5: establishing a PA plastic failure prediction model for principal component analysis (PCA) based on characteristic parameters of the acoustic emission signal, where the model is configured to evaluate a degree of failure and destruction of a material and to predict a failure occurring time.

S6: predicting a degree of failure and destruction of the PA plastic sample and a failure occurring time by using the PA plastic failure prediction model.

### Material and Preparation:

The material for the experiment is polyamide (PA). 25 dumbbell-shaped PA stretching test samples are meticulously prepared in accordance with the China national standard GB/T 1040-2006 and the international standard ISO 3167-2002 [15]. In this research, a sample of the 1BA model is used, an overall length of which is determined to be 100 mm based on an original size of the raw material.

### Main Instrument and Device:

a microcomputer control electronic universal testing machine, with a model of CMT5504/50KN and bundled software of SANS-PowerTest; an acoustic emission and vibration-integrated monitor for low-speed load bearing components, with a model of SAEU3H and bundled software of SWAE; and a sensor of G150-2. Parameter settings of the acoustic emission collection system are shown in Table 1.

TABLE 1
System parameter settings

| Parameters | Numeric value |
|---|---|
| Parameter threshold (dB) | 40 |
| Preamplifier (dB) | 40 |
| Impact definition time (μs) | 2000 |
| Impact locking time (μs) | 20000 |
| Peak definition time (μs) | 1000 |
| FFT sampling decimation rate | 10 |

Experimental scheme: Sample preparation is performed in accordance with a 1BA stretching sample of the ISO3167-2002, with stretching speeds of 0.3 mm/min, 1.0 mm/min, 2.5 mm/min, 5.0 mm/min, and five parallel tests are repeated at each stretching speed. A high-precision acoustic emission monitoring system collects, in real time during a stretching test, an acoustic emission signal generated by the sample under an external load. Parameters of the collected signal include multiple key indicators such as an amplitude, a duration, a ring count, energy, a rise count, a rise time, and a root mean square (RMS), which provide extensive data support for in-depth analysis of internal damage evolution and failure mechanisms of the material.

The collected signal is processed and analyzed through an acoustic emission analysis software, to identify a characteristic and a pattern of the signal. The stretching test is conducted on a SANS-PowerTest universal testing machine with a model of CMT5504/50KN. Experimental parameters of relevant testing instruments are configured as required. Two ends of the sample are fixed through a built-in fixture of the testing machine, a position on the sample for placing a sensor is marked with a marker pen, a coupling agent is applied to a bottom of the sensor, and the sensor and the sample are connected through a tape to ensure accurate capturing of acoustic emission signals. Correlation coefficient analysis is performed on the collected acoustic emission signal to determine an evaluation factor, and then a failure membership degree of each comment set is determined through a triangular membership degree function. Finally, a fuzzy prediction and evaluation model for principal component analysis (PCA) is established based on characteristic parameters of the acoustic emission signal. The model could evaluate a degree of failure and destruction of a material and predict a failure occurring time. A further test is performed to collect data of stretching damage and destruction tests at different speeds, and statistical analysis is performed on characteristic point parameters during damage. The data is compared with a prediction and evaluation result of PCA, and accuracy and feasibility of the prediction model are verified.

### 3. Establishment of a PA Plastic Failure Prediction Model

### 1) Determining of an Evaluation Factor

It is learned from comparisons among the acoustic emission signals during stretching damage of the PA materials at four different stretching speeds that the acoustic emission signal is largely unaffected by the stretching speed. In this research, stretching test data at a speed of 0.3 mm/min is used as a basis for analysis. A series of key characteristic parameters, including an amplitude, a duration, a ring count, energy, a rise count, a rise time, and a root mean square (RMS) are collected through meticulous measurement of the acoustic emission signals of the polyamide (PA) material during stretching. In order to use these parameters to perform fussy prediction on a degree of failure of the plastic, a linear correlation between the acoustic emission signal evaluation factors is first evaluated in this disclosure. A correlation coefficient between the evaluation factors is obtained through statistical analysis on experimental data by using a Corrcoef function in MATLAB. An analysis result shows that a correlation among the amplitude, the ring count, and the rise time is weak (see Table 2 for details), while a correlation among the duration, the energy, the rise counts, and the RMS is strong, which therefore could not be used as independent fuzzy prediction and evaluation factors. Instead, the amplitude, the ring count, and the rise time are determined as evaluation factors.

TABLE 2
Analysis results of correlation coefficients among
the amplitude, the ring count, and the rise time

| Item | Amplitude | Ring count | Rise time |
|---|---|---|---|
| Amplitude | 1 | −0.395410601 | 0.01445782 |
| Ring count | −0.395410601 | 1 | −0.384762578 |
| Rise time | 0.01445782 | −0.384762578 | 1 |

Definitions and uses of the three parameters are shown in Table 3.

TABLE 3
Acoustic emission characteristic parameters

| Parameters | Definition | Characteristic and use |
|---|---|---|
| Amplitude | It refers to a maximum | It is an important parameter in a |
| | amplitude value of a | coustic emission detection as it |
| | signal waveform, | has a direct relationship with a |
| | usually expressed in | magnitude of an event. It is not |
| | decibels (dB). | affected by a threshold and |
| | | could directly determine |
| | | measurability of an event. |
| Ring count | It refers to the number | It is used to evaluate activity of |
| | of waveform oscillations | an acoustic emission event and |
| | after a signal exceeds a | intensity of a signal, is sensitive |
| | set threshold. | to a threshold setting, and is |
| | | often used to evaluate acoustic |
| | | emission activity. |
| Rise time | It refers to a time from | It is an important parameter of |
| | the starting to a peak | characteristics of an acoustic |
| | value of a signal. | emission signal, which could |
| | | manifest a dynamic characteris- |
| | | tic of an acoustic emission |
| | | source, and is used to distinguish |
| | | between acoustic emission |
| | | events of different types. |

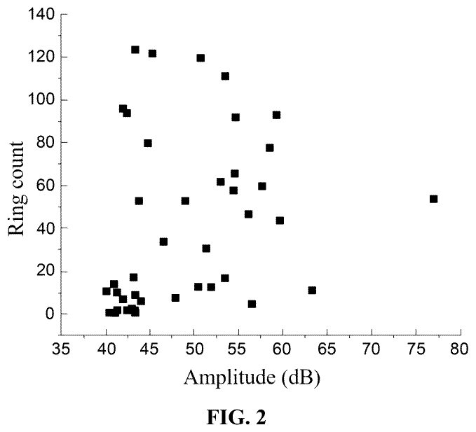

A diagram of an association between the amplitude and the ringing count obtained from the PA sample acoustic emission monitoring is shown in FIG. 2. It may be learned from the diagram of the association between the amplitude and the ring count that the PA almost produces no AE signal in an elastic stage. During stretching, the sample begins to deform. In an amplitude interval (40, 45), the acoustic emission source is relatively active, a small number of acoustic emission signals concentrate, indicating that the PA is in a minor failure stage. In an amplitude interval (45, 60), the PA is in a moderate failure stage, and movements of inner segments (amorphous regions) of the material begins to increase. In an amplitude interval (60, 80), the PA is in a severe failure stage, and a large number of microcrystalline macromolecules begin to slide, causing fine neck deformation. Under further application of the stretching load, the flexible material reaches a pressure bearing capacity extremity and eventually fractures, and thereby a fracture point signal appears.

### 2) Determining of a Weight

According to the basic theory of principal component analysis (PCA), a single principal component may have redundant information between multiple evaluation indicators incorporated therein. The information superposition results in significant differences in contributions of different principal components in interpreting data variability. Because dimensions and numerical ranges of different variables in the original data may differ, standardization is required to avoid impact of indicator dimensions. During stretching, parametric analysis is performed by using relevant parameters collected by an acoustic emitter, and the data is standardized by using a principal component analysis method, to eliminate a dimensional difference and achieve zero-mean normalization. Principal components are selected based on magnitudes of characteristic values, and a projection matrix is established to map the original data onto these principal components, to achieve dimensionality reduction of the data. Sample data is standardized according to an equation (1) to eliminate influence of a dimensional difference in acoustic emissions:

$${X}_{i j}^{*} = \frac{{x}_{i j} - {\overline{x}}_{j}}{{σ}_{j}} ( i = 1 , 2 , … , n ; j = 1 , 2 , … , p ) . \qquad equation ( 1 )$$

$${X}_{i j}^{*}$$

represents a normalized parameter, xᵢⱼ represents an original parameter, x̅ⱼ represents a sample mean, and σⱼ represents a sample standard deviation.

An original data matrix Y is transposed to obtain a transposed matrix Y^{T}, relevant variables in an original dataset are converted into a set of few independent variables, where these new variables become principal components, and a variance contribution and a cumulative contribution of each principal component are calculated. Under the condition that the cumulative contribution is greater than 85%, k principal components are determined. The principal components are characterized by using a new indicator y₁, y₂, . . . , yₙ:

$${ε}_{j} = {a}_{1 j} {y}_{1} + {a}_{2 j} {y}_{2} + … + {a}_{nj} {y}_{n} ( j = 1 , 2 , … , k ) \qquad equation ( 2 )$$

εⱼ represents a weight of each principal component, and aₙⱼ represents a principal component coefficient. The calculation provides a comprehensive score of the new sample, which is then normalized. A result of the processing is used as a weight of each indicator in the original data matrix for analysis. For a large amount of data collected in the experiment, the data is normalized, and then weights of various evaluation factors including the amplitude, the ring count, and the rise time are determined from the data to provide accurate weight basis for subsequent data analysis and evaluation.

### 3) Determining of a Membership Degree Function

Establishing the membership degree function is vital to fuzzy evaluation and prediction, which quantizes an association between an acoustic emission signal and a specific damage level. In the stretching test, a degree of damage of the acoustic emission signal is divided into three levels, i.e., minor damage (a yield stage), moderate damage (a neck reduction stage), and severe damage (a fracture stage), to provide accurate metrics for evaluation of material damage.

In the minor failure stage, the amplitude, the ring count, and the rise time are respectively 40.1-47.9, 1-2, and 1-111; in the moderate failure stage, the amplitude, the ring count, and the rise time are respectively 47.9-60.6, 2-14, and 11-31; and in the severe failure stage: the amplitude, the ring count, and the rise time are respectively >60.6, >14, and >31. In the minor failure stage, the sample is subjected to stretching yield, in which case fracturing or dislocation of polymer chains is particularly significant. Therefore, event count data in this stage is significantly higher than that in the moderate failure region. Stretching deformation in the moderate failure stage is relatively smooth and an event count is relatively small. However, in the severe failure stage, the event count increases sharply again as the polymer chains fracture sharply, indicating intensification of material damage.

### 3-1) Determining of a Membership Degree Function for the Amplitude

Under the condition that a correlation between the acoustic emission signal and a degree of damage during PA stretching is precisely depicted by using a triangular membership degree function, membership degree functions of an amplitude characteristic parameter at three levels, i.e., minor failure, moderate failure, and severe failure are respectively defined as u₁(x), u₂(x), and u₃(x),

$${u}_{1} ( x ) = \{ \begin{matrix}1 , x \leq 40.1 \\ \frac{x - 40.1}{47.9 - 40.1} , 40.1 < x \leq 47.9 \\ \frac{60.6 - x}{60.6 - 47.9} , 47.9 < x \leq 60.6 \\ 0 , x \geq 60.6\end{matrix}$$

$${u}_{2} ( x ) = \{ \begin{matrix}0 , x \leq 40.1 \\ \frac{47.9 - x}{47.9 - 40.1} , 40.1 < x \leq 47.9 \\ \frac{x - 47.9}{60.6 - 47.9} , 47.9 < x \leq 60.6 \\ 0 , x > 60.6\end{matrix}$$

$${u}_{3} ( x ) = \{ \begin{matrix}0 , x \leq 60.6 \\ 1 , x > 60.6\end{matrix} .$$

### 3-2) Determining of a Membership Degree Function for the Ring Count

Under the condition that a correlation between the acoustic emission signal and a degree of damage during PA stretching is precisely depicted by using a triangular membership degree function, membership degree functions of a ring count characteristic parameter at three levels, i.e., minor failure, moderate failure, and severe failure are respectively defined as v₁(x), v₂(x), and v₃(x),

$${v}_{1} ( x ) = \{ \begin{matrix}1 , x \leq 1 \\ \frac{x - 1}{2 - 1} , 1 < x \leq 2 \\ \frac{14 - x}{14 - 2} , 2 < x \leq 14 \\ 0 , x \geq 14\end{matrix}$$

$${v}_{2} ( x ) = \{ \begin{matrix}0 , x \leq 1 \\ \frac{2 - x}{2 - 1} , 1 < x \leq 2 \\ \frac{x - 2}{14 - 2} , 2 < x \leq 14 \\ 0 , x \geq 14\end{matrix}$$

$${v}_{3} ( x ) = \{ \begin{matrix}0 , x \leq 14 \\ 1 , x > 14\end{matrix} .$$

### 3-3) Determining of a Membership Degree Function for the Rise Time

Under the condition that a correlation between the acoustic emission signal and a degree of damage during PA stretching is precisely depicted by using a triangular membership degree function, membership degree functions of a rise time characteristic parameter at three levels, i.e., minor failure, moderate failure, and severe failure are respectively defined as w₁(x), w₂(x), and w₃(x),

$${w}_{1} ( x ) = \{ \begin{matrix}0 , x \leq 1 \\ \frac{x - 1}{11 - 1} , 1 < x \leq 11 \\ \frac{31 - x}{14 - 11} , 11 < x \leq 31 \\ 0 , x \geq 31\end{matrix}$$

$${w}_{2} ( x ) = \{ \begin{matrix}0 , x \leq 1 \\ \frac{11 - x}{11 - 1} , 1 < x \leq 11 \\ \frac{x - 11}{31 - 11} , 11 < x \leq 31 \\ 0 , x \geq 31\end{matrix}$$

$${w}_{3} ( x ) = \{ \begin{matrix}0 , x \leq 31 \\ 1 , x > 31\end{matrix} .$$

### Fuzzy Prediction and Evaluation of a Degree of PA Failure

A series of verification experiments are conducted to ensure accuracy of the fuzzy prediction model. Stretching damage experiments are conducted on four polyamide (PA) samples numbered #1, #2, #3, and #4 under four different stretching speeds ranging from 0.3-5.0 mm/min, with each numbering representing a specific stretching speed. During the experiments, the entire stretching damage process is monitored in real time by using an acoustic emission monitoring device, from which a comprehensive acoustic emission signal characteristic parameter is extracted for subsequent analysis.

### 1) Establishment of an Evaluation Set and a Factor Set

The evaluation set is X=(X₁, X₂, X₃) (minor failure, moderate failure, and severe failure), and the factor set is Y=(Y₁, Y₂, Y₃) (the amplitude, the ring count, and the rise time).

### 2) Determining of a Weight Set

The weight set is A=(A₁, A₂, A₃)=(0.30, 0.41, 0.29).

### (3) Establishment of an Evaluation Matrix and Comprehensive Fuzzy Evaluation

A failure-degree membership of each evaluation point and a fuzzy comprehensive evaluation model B=A·R (B represents an evaluation vector, and R represents a correlation coefficient matrix) are determined according to the membership degree function, and the evaluation vector and the correlation coefficient matrix are used as an input of the model to calculate a comprehensive failure evaluation matrix of each evaluation point. The evaluation matrix is used to perform comprehensive fussy prediction and evaluation on a degree of damage of each evaluation point.

$$R = \begin{matrix}{r}_{11} & {r}_{12} & {r}_{13} \\ {r}_{21} & {r}_{22} & {r}_{23} \\ {r}_{31} & {r}_{32} & {r}_{33}\end{matrix} ,$$

where r₁₁ represents a failure membership degree of the amplitude at the level of minor failure, r₁₂ represents a failure membership degree of the amplitude at the level of moderate failure, r₁₃ represents a failure membership degree of the amplitude at the level of severe failure, r₂₁ represents a failure membership degree of the ring count at the level of minor failure, r₂₂ represents a failure membership degree of the ring count at the level of moderate failure, r₂₃ represents a failure membership degree of the ring count at the level of severe failure, r₃₁ represents a failure membership degree of the rise time at the level of minor failure, r₃₂ represents a failure membership degree of the rise time at the level of moderate failure, and r₃₃ represents a failure membership degree of the rise time at the level of severe failure.

Statistical analyses are conducted on characteristic parameters including amplitudes, ringing counts, and rise times of acoustic emission signals of three points during stretching damage of the PA sample, and statistical results are shown in Table 4.

TABLE 4
Characteristic parameters of acoustic emission signals
in the PA damage process at four stretching speeds

| Sample No. | Time point/s | Amplitude/dB | Ring count | Rise time |
|---|---|---|---|---|
| #1 | Point 1 (121.0) | 45.3 | 6.0 | 2.0 |
| | Point 2 (347.0) | 60.6 | 11.0 | 4.0 |
| | Point 3 (542.1) | 46.5 | 99.0 | 496.0 |
| #2 | Point 1 (8.0) | 43.3 | 9.0 | 32.0 |
| | Point 2 (42.0) | 43.1 | 17.0 | 57.0 |
| | Point 3 (52.1) | 53.5 | 111.0 | 213.0 |
| #3 | Point 1 (3.0) | 67.9 | 113.0 | 696.0 |
| | Point 2 (3.7) | 56.7 | 69.0 | 1145.0 |
| | Point 3 (49.7) | 52.5 | 18.0 | 31.0 |
| #4 | Point 1 (0.5) | 43.5 | 2.0 | 6.0 |
| | Point 2 (2.7) | 45.0 | 8.0 | 10.0 |
| | Point 3 (5.1) | 87.5 | 174.0 | 1587.0 |

Failure membership degrees of the PA samples numbered #1, #2, #3, and #4 at each time point are calculated according to the membership degree function based on the statistical analysis of acoustic emission characteristic parameters of the polyamide (PA) plastic sample at different damage stages and acoustic emission data collected at three key time points. Taking the membership calculation of the sample numbered #1 at the first time point as an example (as shown below), the membership calculation for the other samples being same as that for the sample #1, the calculation result is shown in Table 5.

TABLE 5
Failure membership degree of a PA sample #1 at a point 1

| Failure evaluation | Amplitude | Ring count | Rise time |
|---|---|---|---|
| Minor failure | 0.67 | 0.67 | 0.10 |
| Moderate failure | 0.31 | 0.33 | 0.90 |
| Severe failure | 0.00 | 0.00 | 0.00 |

A shown in Table 5, an evaluation vector of the PA sample #1 at the point 1 is calculated as follows:

$${B}_{1} = A \cdot {R}_{1} = [ \begin{matrix}0.3 & 0.41 & 0.29\end{matrix} ] [ \begin{matrix}0.67 & 0.31 & 0 \\ 0.67 & 0.33 & 0 \\ 0.1 & 0.9 & 0\end{matrix} ] = [ \begin{matrix}0.51 & 0.49 & 0\end{matrix} ]$$

During failure analysis of the polyamide (PA) plastic, vector data correspond to the minor failure, moderate failure, and severe failure stages of the material, so that a degree of damage at the point 1 could be evaluated. For evaluation points 2 and 3, corresponding evaluation vectors are respectively B2=[0.19 0.81 0] and B3=[0.25 0.05 0.7], which are also suitable for evaluating degrees of failure at the two points. Similarly, for PA plastic samples, including the samples numbered #2, #3, and #4, the corresponding evaluation vectors may also be used to evaluate a damage state at each evaluation point.

Evaluation results are as follows: During the comprehensive evaluation and analysis of the polyamide (PA) sample #1, degrees of failure at three key evaluation points are determined. Specifically, a comprehensive evaluation vector at the point 1 is [0.51 0.49 0], which is classified as minor failure according to a maximum membership principle. A comprehensive evaluation vector at the point 2 is [0.19 0.81 0], indicating that the point is in a moderate failure state. A comprehensive evaluation vector at the point 3 is [0.25 0.05 0.7], indicating a more severe failure level. These results provide an in-depth understanding of the failure behaviors of the PA samples at different stretching stages.

In this disclosure, a series of stretching destruction experiments are conducted on the polyamide (PA) plastic samples by using a universal testing machine, which involves four different stretching speeds. During the experiments, detailed collection and analysis are conducted on the acoustic emission signals generated in the stretching damage process of the samples, and a fuzzy prediction evaluation model for principal component analysis (PCA) based on acoustic emission is established, to evaluate a degree of failure and destruction of the PA plastic during stretching. The model is established to provide a new analysis method for accurately predicting a failure behavior of a material, which could determine a degree of failure of a sample at any time point, effectively monitor and evaluate the failure and destruction process of the PA plastic at different stretching speeds in real time, and provide scientific basis for engineering design and application of materials.

1. A polyamide (PA) plastic failure prediction method, comprising steps of:
preparing a PA plastic sample, and configuring a stretching speed and a number of stretches;
collecting, by an acoustic emission monitoring system, in real time during a stretching test, an acoustic emission signal generated by the PA plastic sample under an external load;
processing and analyzing collected acoustic emission signal through an acoustic emission analysis software, to identify a characteristic and a pattern of the acoustic emission signal;
performing correlation coefficient analysis on the collected acoustic emission signal to determine evaluation factors, and then determining a failure membership degree function of each evaluation factor through a triangular membership degree function;
establishing a PA plastic failure prediction model for principal component analysis based on characteristic parameters of the acoustic emission signal, wherein the PA plastic failure prediction model is configured to evaluate a degree of failure and destruction of a material;
acquiring, by an acoustic emission sensor arranged in a hydrogen cylinder liner made of the PA plastic sample, values of the evaluation factors of the hydrogen cylinder liner;
determining failure membership degrees of the evaluation factors based on the values of the evaluation factors through the failure membership degree functions; and
inputting the failure membership degrees into the PA plastic failure prediction model to obtain a prediction result, wherein the prediction result indicates a degree of failure and destruction of the hydrogen cylinder liner.

2. The PA plastic failure prediction method as claimed in claim 1, further comprising:
performing a further test to collect data of stretching damage and destruction tests at different stretching speeds, performing statistical analysis on parameters at characteristic points during damage, comparing the data with an evaluation result of the PA plastic failure prediction model, and verifying the PA plastic failure prediction model.

3. The PA plastic failure prediction method as claimed in claim 1, wherein the PA plastic sample is prepared in accordance with a 1BA stretching sample of the ISO3167-2002, with stretching speeds of 0.3 mm/min, 1.0 mm/min, 2.5 mm/min, 5.0 mm/min, wherein five parallel tests are repeated at each stretching speed; and parameters of the collected acoustic emission signal comprise an amplitude, a duration, a ring count, energy, a rise count, a rise time, and a root mean square (RMS).

4. The PA plastic failure prediction method as claimed in claim 1, wherein the PA plastic failure prediction model is established by: determining an evaluation factor of a fuzzy prediction and evaluation model;
configuring a weight of the evaluation factor in the fuzzy prediction and evaluation model; and
determining a membership degree function for the evaluation factor, and establishing the PA plastic failure prediction model through the weight of the evaluation factor and the membership degree function for the evaluation factor.

5. The PA plastic failure prediction method as claimed in claim 4, wherein an amplitude, a ring count, and a rise time of the acoustic emission signal are determined as the evaluation factor of the fuzzy prediction and evaluation model.

6. The PA plastic failure prediction method as claimed in claim 4, further comprising
standardizing sample data according to equation (1) to eliminate influence of a dimensional difference in acoustic emissions:
$${X}_{i j}^{*} = \frac{{x}_{i j} - {\overline{x}}_{j}}{{σ}_{j}} ( i = 1 , 2 , … , n ; j = 1 , 2 , … , p ) , \qquad equation ( 1 )$$
wherein
$${X}_{i j}^{*}$$
represents a standardized parameter, xᵢⱼ represents a j-th evaluation factor in a i-th sample comprising respective evaluation factors acquired at a corresponding moment, x̅ⱼ represents a mean of the j-th evaluation factor, and σⱼ represents a standard deviation of the j-th evaluation factor; and
transposing an original data matrix Y composed of the standardized parameter
$${X}_{ij}^{*}$$
to obtain a transposed matrix Y^{T}, calculating a correlation coefficient matrix R of the original data matrix Y and in turn eigenvalues λ₁, λ₂ . . . λ_{P} and eigenvectors u₁, u₂ . . . u_{P} of the correlation coefficient matrix R, converting relevant variables in an original dataset into a set of independent variables, where these new variables become principal components, and calculating a variance contribution and a cumulative contribution of each principal component, wherein under the condition that the cumulative contribution is greater than 85%, k principal components are determined:
$${ε}_{j} = \underline{{a}_{1} {y}_{1}} + \underline{{a}_{2 j} {y}_{2}} + … + \underline{{a}_{kj} {y}_{k}} , \qquad equation ( 2 )$$
wherein
εⱼ represents a comprehensive score of the j-th evaluation factor, yₖ represents a variance contribution rate of a k-th principal component and expressed as
$${y}_{k} = \frac{{λ}_{i}}{{\sum }_{i = 1}^{p} {λ}_{i}} ,$$
and aₖⱼ represents a coefficient or a load of the j-th evaluation factor in the k-th principal component and is expressed as aₖⱼ=\sqrt{λₖ}×uₖⱼ, where uₖⱼ denotes a j-th element in an eigenvector uₖ; and
obtaining a comprehensive score of a new sample through calculation above, then normalizing, and using a normalized result as a weight of each indicator in the original data matrix, and for a large amount of data collected during test(s), normalizing the data, and determining, from a normalized result, weights of evaluation factors of an amplitude, a ring count, and a rise time.

7. The PA plastic failure prediction method as claimed in claim 4, wherein a membership degree function for an amplitude is as follows:
under the condition that a correlation between the acoustic emission signal and a degree of damage during PA stretching is depicted by using a triangular membership degree function, membership degree functions of the amplitude at three levels of minor failure, moderate failure, and severe failure are defined respectively as u₁(x), u₂(x), and u₃(x),
$${u}_{1} ( x ) = \{ \begin{matrix}1 , x \leq 40.1 \\ \frac{x - 40.1}{47.9 - 40.1} , 40.1 < x \leq 47.9 \\ \frac{60.6 - x}{60.6 - 47.9} , 47.9 < x \leq 60.6 \\ 0 , x \geq 60.6\end{matrix}$$
$${u}_{2} ( x ) = \{ \begin{matrix}0 , x \leq 40.1 \\ \frac{47.9 - x}{47.9 - 40.1} , 40.1 < x \leq 47.9 \\ \frac{x - 47.9}{60.6 - 47.9} , 47.9 < x \leq 60.6 \\ 0 , x > 60.6\end{matrix}$$
$${u}_{3} ( x ) = \{ \begin{matrix}0 , x \leq 60.6 \\ 1 , x > 60.6\end{matrix} .$$

8. The PA plastic failure prediction method as claimed in claim 4, wherein a membership degree function for a ring count is as follows:
under the condition that a correlation between the acoustic emission signal and a degree of damage during PA stretching is depicted by using a triangular membership degree function, membership degree functions of the ring count at three levels of minor failure, moderate failure, and severe failure are respectively defined as v₁(x), v₂(x), and v₃(x),
$${v}_{1} ( x ) = \{ \begin{matrix}1 , x \leq 1 \\ \frac{x - 1}{2 - 1} , 1 < x \leq 2 \\ \frac{14 - x}{14 - 2} , 2 < x \leq 14 \\ 0 , x \geq 14\end{matrix}$$
$${v}_{2} ( x ) = \{ \begin{matrix}0 , x \leq 1 \\ \frac{2 - x}{2 - 1} , 1 < x \leq 2 \\ \frac{x - 2}{14 - 2} , 2 < x \leq 14 \\ 0 , x \geq 14\end{matrix}$$
$${v}_{3} ( x ) = \{ \begin{matrix}0 , x \leq 14 \\ 1 , x > 14\end{matrix} .$$

9. The PA plastic failure prediction method as claimed in claim 4, wherein a membership degree function for a rise time is as follows:
under the condition that a correlation between the acoustic emission signal and a degree of damage during PA stretching is depicted by using a triangular membership degree function, membership degree functions of the rise time characteristic parameter at three levels of minor failure, moderate failure, and severe failure are respectively defined as w₁(x), w₂(x), and w₃(x),
$${w}_{1} ( x ) = \{ \begin{matrix}0 , x \leq 1 \\ \frac{x - 1}{11 - 1} , 1 < x \leq 11 \\ \frac{31 - x}{14 - 11} , 11 < x \leq 31 \\ 0 , x \geq 31\end{matrix}$$
$${w}_{2} ( x ) = \{ \begin{matrix}0 , x \leq 1 \\ \frac{11 - x}{11 - 1} , 1 < x \leq 11 \\ \frac{x - 11}{31 - 11} , 11 < x \leq 31 \\ 0 , x \geq 31\end{matrix}$$
$${w}_{3} ( x ) = \{ \begin{matrix}0 , x \leq 31 \\ 1 , x > 31\end{matrix} .$$

10. The PA plastic failure prediction method as claimed in claim 4, wherein a fuzzy prediction and evaluation of a degree of PA failure is performed by a process comprising steps of:
1) establishing an evaluation set and a factor set, wherein
the evaluation set is X=(X₁, X₂, X₃), and the factor set is Y=(Y₁, Y₂, Y₃), wherein
X₁ represents minor failure, X₂ represents moderate failure, X₃ represents severe failure, Y₁ represents an amplitude, Y₂ represents a ring count, and Y₃ represents a rise time;
2) determining a weight set, wherein
the weight set is A=(A₁, A₂, A₃)=(0.30, 0.41, 0.29); and
(3) establishing an evaluation matrix and comprehensive fuzzy evaluation, wherein
according to the membership degree function, determining a membership degree in terms of failure degree of each time point and a fuzzy comprehensive evaluation model B=A·R, wherein B represents an evaluation vector, and R represents a correlation coefficient matrix, and the evaluation vector and the correlation coefficient matrix are configured as an input of the PA plastic failure prediction model to calculate a comprehensive failure evaluation matrix of each time point.

11. The PA plastic failure prediction method as claimed in claim 1, further comprising:
in response to determining that the prediction result indicates that the degree of failure and destruction of the hydrogen cylinder liner is a severe failure, determining a time at which the values of the evaluation factors corresponding to the prediction result are acquired as a failure occurring time.

12. The PA plastic failure prediction method as claimed in claim 11, further comprising:
determining a residual lifetime of the hydrogen cylinder liner by using an equation as follow
$$R L = \frac{{T}_{f} - {T}_{o}}{S} ,$$
where RL is the residual lifetime of the hydrogen cylinder liner, S is a security coefficient that is 1.5, T_{f} is the failure occurring time, and Tₒ is a current running time of the hydrogen cylinder liner.

13. The PA plastic failure prediction method as claimed in claim 1, further comprising:
replacing the hydrogen cylinder liner in response to the degree of failure and destruction of the hydrogen cylinder liner being in a severe failure.
